(19) United States
(12) Patent Application Publication
(10) Pub. No.: US 2026/0272694 A1
(43) Pub. Date: Sep. 17, 2026

(54) VACUUM SPLINT DRESSING WITH GRANULE FILLING

(71) Applicant: Bo RENBERG, Degerfors (SE)

(72) Inventor: Bo RENBERG, Degerfors (SE)

(21) Appl. No.: 19/480,295

(22) PCT Filed: Apr. 30, 2024
(86) PCT No.: PCT/SE2024/050413

§ 371 Date: Oct. 30, 2025

(30) Foreign Application Priority Data
May 2, 2023 (SE) 2330192-2

(51) Int. Cl. A61F 5/058

(52) U.S. Cl. CPC A61F 5/05833; A61F 5/0585; A61F 5/05866

(57) ABSTRACT

Vacuum splint dressing (1) comprising a multilayer laminate (2) forming an airtight volume partially filled with a filler of granules, wherein an evacuation and check valve (12) forming a sealable passage for air through one of the laminate layers, the granules consisting of bodies (20; 21) of expanded polypropylene (EPP).

### TECHNICAL FIELD OF THE INVENTION

The present invention relates to a vacuum splint dressing comprising a multilayer laminate which forms an airtight volume partially filled with a filler of granules, wherein an evacuation and check valve forms a sealable passage for air through one of the laminate layers.

### BACKGROUND AND PRIOR ART

Splint dressings of this type are previously known and described in the patent literature, see for example SE525021C2, WO2004/082544A1 and U.S. Pat. No. 6,918,393B2.

The efficiency of these vacuum splint dressings relies, in short, on the fact that the friction in a granule filling between airtight outer material layers is increased by creating negative pressure in the granule-filled volume, whereby the granule forms a rigid body. At normal air pressure, the granules return to a loose and almost liquid state that can be formed around an injured joint, limb or torso in order to stabilize the injured body part when the granules are put under negative pressure. It should be understood that the lower the air pressure that can be created in the vacuum splint dressing, the harder the granule bodies are locked together. The negative pressure is typically created by connecting a hand-operated pump to a valve incorporated in the vacuum splint dressing. Although complete vacuum is unlikely to be achieved by manually pumping out the air, it may be justified to call these items vacuum splint dressings to distinguish them from plaster casts or splint bandage with integral laths, for example.

Existing vacuum splint dressings are typically used for temporary stabilization of an injured body part, such as during transport from an accident scene to a medical facility. After that, a more permanent stabilization can be applied around the body part, usually in the form of a plaster cast.

One reason why existing vacuum splint dressings are usually not used for a longer period of time, such as during healing and during rehabilitation, is that the granule bodies change their shape after a period of negative pressure or due to external influences. Thereby, the total volume of the granule filling between the airtight outer layers is reduced, with the result that the splint dressing softens. The decreasing hardness of the splint dressing is a disadvantage during longer transports and reduces the use of existing vacuum splint dressings to temporary applications that are not suitable for application during a healing process or for rehabilitation purposes.

### SUMMARY OF THE INVENTION

In view of the background described above, it is an object of the invention to provide a vacuum splint dressing that maintains vacuum, or the effective negative pressure in an applied vacuum splint dressing, for a longer time to enable longer transports and use during healing/rehabilitation.

Another object is to provide a vacuum splint dressing that can be applied and removed by the patients themselves during healing and rehabilitation.

Yet another object is to provide a vacuum splint dressing that can remain applied when showering and bathing during healing and rehabilitation.

A further object is to provide a vacuum splint dressing that can be equipped with accessories to facilitate the patient's everyday life and/or to extend the function and use of the vacuum splint dressing.

One or more of these objects is fulfilled by a vacuum splint dressing of the type indicated in the above introduction, wherein the granule consists of bodies of expanded polypropylene with long-term shape memory.

Granule bodies of the aforementioned materials can be formed with a favorable balance between weight and dimensional stability, so that for a long time, such as for several days up to 4-6 weeks or longer, they resist being deformed under effective negative pressure so that the vacuum splint dressing becomes light and flexible and thus not strainingly heavy for the patient to wear.

In one embodiment of the invention, the granule bodies are made of expanded polypropylene with an original density in the order of about 900 g/L, which is expanded at a ratio of about 1/10 to about 1/20, preferably about 1/15, resulting in a density of about 90 g/L to about 45 g/L.

Granule bodies of this embodiment exhibit high resistance to deformation and enable continuous use of the vacuum splint dressing, even for a longer period of time such as during a healing process or during a rehabilitation period, without losing its shape or changing its properties under changing pressure conditions. An advantage of the preferred granule is that granule bodies of expanded polypropylene have a closed cell structure and do not absorb water or other chemicals and have great resistance to temperature variations, so that the vacuum splint dressing maintains its properties under both cold and heat conditions. Granule bodies made of expanded polypropylene EPP also prevent the occurrence of mould and bacteria.

Granule bodies of the aforementioned embodiment have also been shown to exhibit long-term shape memory. This property has, through practical tests, proven to be very valuable in a vacuum splint dressing intended for use by a patient for a longer period of time, when the granule bodies are exposed to repeated pressure changes when applying and removing the vacuum splint dressing. The preferred granule body has thereby exhibited a very stable shape even after a longer period of use.

In a preferred embodiment of the invention, the granule bodies consist of expanded polypropylene with a density in the order of about 45 g/L to about 55 g/L, with an average density of about 50 g/L.

Granule bodies of this embodiment exhibit high resistance to deformation despite comparatively low weight, and may be particularly suitable in a vacuum splint dressing to be worn by children, weak-muscled or elderly persons.

One embodiment of the invention includes granule bodies of expanded polypropylene with a density at the upper end of the range or about 55 g/L.

Granule bodies of this embodiment exhibit high resistance to deformation and may be particularly suitable in a vacuum splint dressing to be worn by stronger or heavier adult patients who are active and in motion even during rehabilitation.

An embodiment of the invention includes granule bodies of expanded polypropylene with a density higher than about 55 g/L, within the range of about 55 g/L to about 90 g/L.

Granule bodies of this embodiment exhibit a very high resistance to deformation, and may be particularly suitable in a vacuum splint dressing to be worn by stronger or heavier adult patients who are active and in motion even during rehabilitation, such as athletes or patients undergoing physical therapy during the time they wear the vacuum splint dressing.

An advantage of all embodiments of the vacuum splint dressing according to the invention is that it can be removed and re-applied by the patients themselves or by home nursing staff or at any care institution in a way that cannot be done with a conventional plaster cast, while in the applied position it provides fixation of an injured body part substantially to the same degree as a plaster cast.

According to one embodiment of the invention the granule filling in the multilayer laminate consists of non-spherical bodies in the order of approx. 1.5 mm to approx. 3 mm.

Non-spherical granule bodies lock together and form a harder core in the multilayer laminate under negative pressure. The preferred size of the granule bodies facilitates the forming of the vacuum splint dressing around a body part before the air is sucked out of the multilayer laminate.

Other embodiments of the invention include spherical granule bodies in the order of approx. 1.5 mm to approx. 3 mm.

In one embodiment, the multilayer laminate comprises a first airtight surface layer of plastic, preferably made of polyurethane, and a second airtight surface layer of plastic, preferably made of polyurethane, and an intermediate air-permeable layer, preferably in the form of a perforated cloth or fabric made of nylon/polyamide or polyester, which is joined to the respective surface layer by means of a welded connection.

The preferred materials in the multilayer laminate are environmentally friendly and chosen to avoid skin irritation in the wearer.

In a preferred embodiment, the surface layers and the intermediate permeable layer are joined together by a doubled welded connection consisting of two welded joints which run in parallel along the peripheries of the vacuum splint dressing.

The doubled welded joint ensures an airtight connection of the layers in the multilayer laminate. The parallel-running welded joints thus guarantee double airtight barriers around the entire periphery of the multilayer laminate.

An embodiment of the vacuum splint dressing includes at least one tightening strap running from either of the polyurethane layers, on which tightening strap is arranged a piece of Velcro tape which is attached to the tightening strap through a welded connection, wherein said welded connection runs around the Velcro tape and seals the surface behind the Velcro tape.

Fixing the Velcro tape in this way prevents the ingress of dirt and liquids between the Velcro tape and the tightening strap, which raises the hygienic standard of the vacuum splint dressing.

An embodiment of the vacuum splint dressing has a valve system in the form of an evacuation valve with a check valve function which comprises a valve holder part which is permanently and airtightly anchored in an opening through one of the surface layers, a valve seat part which preferably is removably mountable in the valve holder part by a screw connection, a pump adapter part which preferably is removably mountable in the valve seat part by a screw connection, wherein the valve seat part has a circular seat for a circular valve membrane, wherein the valve membrane is made of thermoplastic elastomer (TPE), preferably polyurethane plastic (TPU).

The valve membrane according to the aforementioned embodiment offers a long service life and has high dimensional stability that ensures sealing for a longer period of time, in favorable cases for weeks or months.

One embodiment of the vacuum splint dressing has at least one additional accessory bracket in the form of a connector accessible on the outside of the multilayer laminate adapted for mounting accessories in addition to the evacuation valve/check valve.

The embodiment provides the opportunity for alternative activities with an applied vacuum splint dressing.

In one embodiment, the accessory bracket is designed as a lead-through provided with a screw thread through one of the surface layers of the multilayer laminate, and is provided with a cover with a seal for airtight closure of the lead-through when accessories are not used.

In one embodiment, the accessory bracket is designed as a lead-through provided with a screw thread through one of the surface layers of the multilayer laminate, wherein an airtight laminate layer that is welded to the inside of said surface layer isolates the lead-through from the volume partially filled with granules of the vacuum splint dressing.

In one embodiment, the vacuum splint dressing is arranged to be equipped with accessories in the form of, e.g.:
a height adjustable heel
a shock-absorbing heel/sole,
a friction-increasing heel/sole,
a fixture brace for a knee or an elbow joint,
a link for connection between adjacent vacuum splint dressings.

### BRIEF DESCRIPTION OF THE DRAWINGS

Embodiments of the invention will now be described in more detail with reference to the attached schematic drawings, which show

FIG. 1 a plan view of an embodiment of a vacuum splint dressing intended for stabilizing a forearm and/or a wrist,

FIG. 2 the vacuum splint dressing of FIG. 1 in the applied position,

FIG. 3 a cross-section of a broken-out part of the vacuum splint dressing of FIG. 1 with an exploded view of the associated check valve,

FIG. 4 an accessory for a vacuum splint dressing,

FIG. 5 another accessory for a vacuum splint dressing according to the invention,

FIG. 6 an application of an accessory in the form of a heel or a sole in a vacuum splint dressing for stabilizing an ankle/ankle joint, and

FIG. 7 an application of an accessory in the form of a stabilization link in a vacuum splint dressing for stabilizing an elbow joint.

### DETAILED DESCRIPTION OF PREFERRED EMBODIMENTS

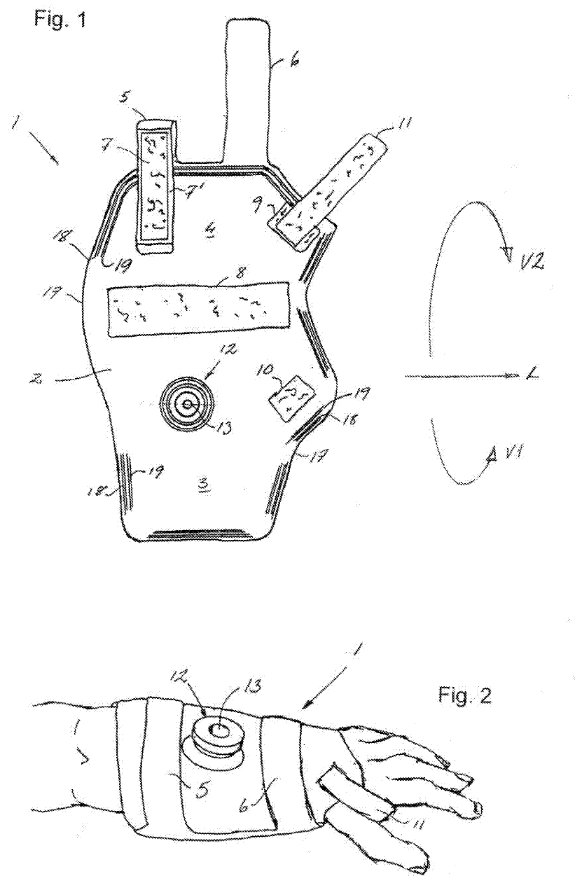

A vacuum splint dressing 1 for stabilizing a forearm and/or a wrist shows, according to FIG. 1, a multilayer laminate 2 consisting essentially of an inner flap 3 that may be applied to the arm and a flap 4 that may be folded over on the outside of the inner flap. From a periphery of the foldable flap, two tightening straps 5 and 6 extend, to one side of which a respective Velcro tape is mounted. In FIG. 1, only the Velcro tape 7 is visible on the tightening strap 5 folded up against the flap 4. Here it should be added that the Velcro tape 7 is attached to the tightening strap 5 by means of a circumferential welded or glue connection (7′) which prevents liquids or particles from getting under the Velcro tape 7.

Furthermore, a Velcro fastener 8 is mounted on the outside of the multilayer laminate. The Velcro fastener 8 extends in the longitudinal direction L of the vacuum splint dressing and its length is adapted so that both tightening straps 5 and 6 may be fastened to the Velcro fastener 8 when the vacuum splint dressing has been applied around the forearm and wrist as illustrated by arrows V1 and V2 in FIG. 1 and by the splinted and stabilized forearm and wrist in FIG. 2. At the end of the vacuum splint dressing 1 facing the hand, two additional Velcro fasteners 9 and 10 are mounted on the multilayer laminate 2. The Velcro fasteners 9 and 10 cooperate with a Velcro tape 11 that runs via the thumb fold and fixes the vacuum splint dressing in the longitudinal direction of the forearm. The Velcro fasteners 8, 9 and 10 may be glued, or welded, to the outside of the multilayer laminate 2. The Velcro fastener 11 may be removably or fixedly mounted in the position of either of the Velcro fasteners 9 or 10.

For evacuating air from the multilayer laminate, a valve 12 is arranged in the multilayer laminate. The valve 12 forms a one-way passage through the outside of the multilayer laminate and in this function constitutes a check valve. The check valve 12 has an externally accessible connection 13 for connecting a pump with which air may be sucked out of the multilayer laminate 2.

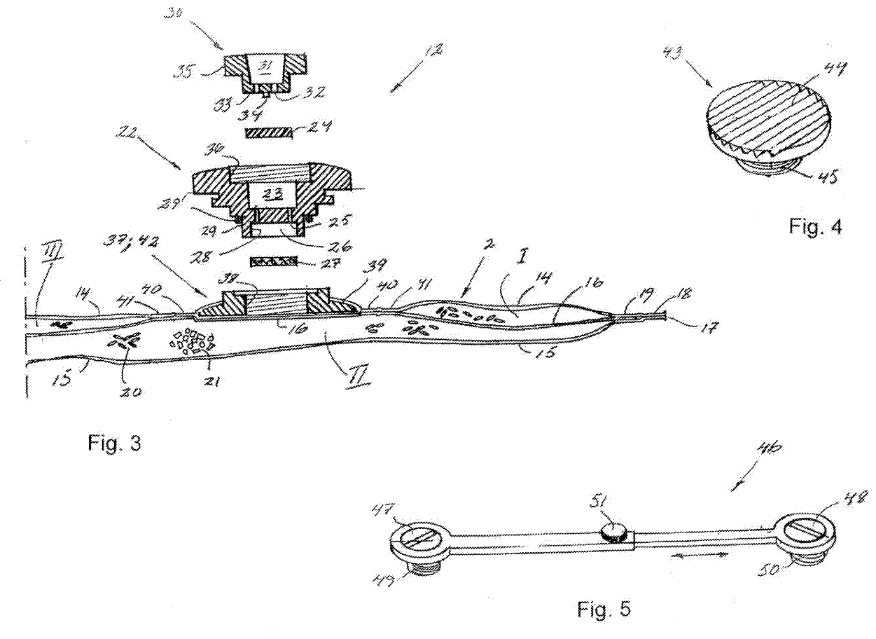

With reference to FIG. 1 and FIG. 3, the structure of the multilayer laminate 2 and the check valve 12 will now be explained in more detail. More specifically, the multilayer laminate 2 comprises a first airtight layer 14 which forms the outside of the multilayer laminate, a second airtight layer 15 which forms the inside of the multilayer laminate which faces the user, and an intermediate air-permeable layer 16. The outer, airtight layers 14 and 15 are of airtight, skin and environmentally friendly and non-allergenic polymer material, rubber or synthetic rubber mixture. A preferred material is a flexible cloth made of polyurethane with or without a plasticizer. The permeable intermediate layer 16 suitably and preferably consists of a perforated cloth, a fabric or a net made of nylon/polyamide or polyester.

The three layers 14, 15 and 16 are connected to each other along the periphery 17 of the multilayer laminate. More specifically, the multilayer laminate 2 is sealed by heat welding which has double welded joints 18 and 19. The welded joints 18 and 19 run in parallel inside the periphery 17 of the multilayer laminate. Although the welded joints 18 and 19 for drawing technical reasons are only partially shown in FIG. 1, it should be understood that they run continuously around the multilayer laminate 2, in parallel inside the periphery 17.

FIG. 3 schematically illustrates how the inner volume of the multilayer laminate is partially occupied by granule bodies 20, 21. The granule bodies may be shaped like rice grains 20 or beans, or have the form of circular discs or rods. The granule bodies can have a spherical shape, but preferably have a non-spherical shape. The granule bodies are suitably shaped as short cylinders 21 with a circular cross-section. These cylinders may have a length in the order of 1 to 5 mm, preferably a length of about 1.5 to 3 mm, and have a diameter in the order of 1 to 3 mm. The granule bodies may also alternatively have an irregular shape and/or an irregular size.

For use in a vacuum splint dressing 1, the granule bodies 20, 21 are preferably made of expanded polypropylene (EPP). EPP is a commercial product that may be obtained in several qualities for different purposes, and for use as granules in a vacuum splint dressing, exhibits particularly advantageous properties such as good energy absorption, deformation resistance, thermal insulation, buoyancy, resistance to water and chemicals. EPP has an exceptionally high strength/weight ratio and is 100 percent recyclable. Granule bodies of EPP can be produced with a wide variation in density, within a range in the order of 15 to 200 g/L. The granule bodies are produced by allowing extruded granule substances to expand into firmly shaped grains of expanded polypropylene. These grains can then be injected into moulds where they are given their final shape under pressure and heat. For the mass production of granule bodies, moulds with a large number of cavities can be used.

As the preferred material for the production of the granule bodies 20 and 21 after very extensive and many years of experimental activity, the applicant has arrived at starting from polypropylene with an original density of about 900 g/L which through a process, such as a foaming process, is expanded in a ratio of about 1/10 to about 1/20, preferably about 1/15 or to have a density of the finished granule body within a density range of about 90 g/L to about 45 g/L.

In a particularly advantageous embodiment, the granule bodies 20 or 21 have a density within a density range in the order of about 45 g/L to about 55 g/L, with an average of about 50 g/L.

FIG. 3 shows the check valve 12 in an exploded, sectional view. The check valve 12 comprises a cylindrical valve seat part 22 with an internal valve seat 23 for a valve membrane 24. The valve membrane 24 is preferably made of a thermoplastic elastomer based on polyurethane, a thermoplastic variant of polyurethane rubber which is often referred to by the abbreviation TPU. A TPU quality suitable for the purpose has a density of about 1 to 1.4 g/cm³ , preferably about 1.2 g/cm³ , and may have a hardness of about 90-95 Shore A (ASTM D 2240). A TPU quality suitable for the purpose can have a breaking strength in the order of about 40 MPa or about 40 N/mm². The valve membrane can have a thickness of approx. 0.5 to approx. 2.5 mm.

The valve membrane according to the aforementioned design may in favorable cases ensure sealing for a longer period of time, such as four to six weeks.

The valve seat part 22 is connected via channels 25 to a filter seat 26 for a filter 27. The filter seat 26 is formed inside a cylindrical wall 28 with an externally threaded outside 29. A pump adapter part 30 includes a pump nozzle seat 31 for removable insertion of a nozzle to a pump. The pump nozzle seat 31 is connected via channels 32 to the valve seat 23. The channels 32 are formed through a bottom 33 of the pump nozzle seat 31, wherein a valve membrane retainer 34 in the underside of said bottom 33 holds down a central area of the valve membrane 24, whereas the peripheral part of the valve membrane may bend upwards when extracting air via the channels 25 and 32. The pump adapter part 30 has an externally threaded outside 35 for engagement with a threaded inside 36 of the valve seat part 22.

The check valve 12 may be mounted in the multilayer laminate 2 by means of a valve holder part 37 which, for this purpose, has a threaded inside 38 which cooperates with the valve seat part 22 threaded on the outside 29.

A seal, preferably an O-ring seal 29′ (see FIG. 3), is arranged to make the connection between the valve seat part 22 and the valve holder part 37 airtight.

According to one embodiment the outside of the valve holder part has a flange 39 with which the valve holder part 37 may be connected airtightly to the outer airtight layer 14 of the multilayer laminate. More specifically, the flange 39 is glued or welded to the inside of the outer layer 14, whereas the intermediate layer 16 is joined to the outer layer 14 by means of double welded joints 40 and 41 which run in parallel around the flange 39.

As can also be seen from FIG. 3, the inner volume of the multilayer laminate may be divided into several chambers I, II, III, which are suitably separated by intermediate permeable laminate layers in order to distribute the granule bodies evenly in the vacuum splint dressing 1 without preventing the passage of air when air is extracted from the multilayer laminate.

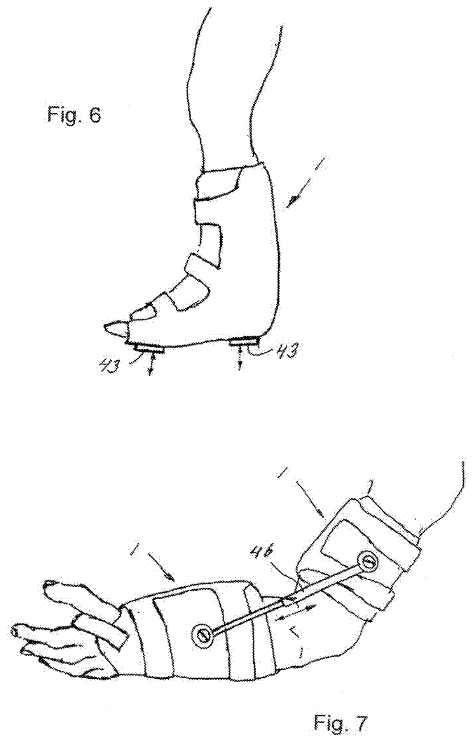

An accessory bracket 42 for mounting accessories on the vacuum splint dressing 1 may be designed similarly to the valve holder part 37 described above. With reference to FIG. 4, an example of an accessory 43 in the form of a plate with a patterned surface 44 is illustrated. An externally threaded pin 45 is dimensioned to be screwed into the accessory bracket 42 (or, as the case may be, into an additional valve holder part 37 which is not used for mounting an evacuation valve/check valve 12). The accessory 43 may be used as a heel or outsole in a vacuum splint dressing that encloses an ankle and a heel, essentially as illustrated in FIG. 6. It should be added that an accessory 43 in the form of a heel or sole may have a different shape from the round shape shown, and that the surface 44 may also have a different pattern from the ribbing shown in FIG. 6. It can also be added that the heel 43 is adjustable in height, as illustrated by the double arrow in FIG. 6, by virtue of the screwing of the threaded pin 45 into the accessory bracket 42. Hereby is provided an adjustable system that allows a vacuum support dressing for the lower leg and ankle to be adjusted to the same height as the other foot to facilitate walking training and speed up rehabilitation.

With reference to FIG. 5, an accessory 46 is shown in the form of a stay or a link 46 which is formed at each end with a screw mount 47, 48 with a respective threaded pin 49 and 50, with which the link 46 may be connected to the respective accessory brackets 42 of two cooperating vacuum splint dressings 1. The link 46 may be telescopically extendable, as illustrated by the double arrow in FIG. 5 and FIG. 7, and be locked at a set length by means of a locking screw 51. As illustrated in FIG. 7, the accessory 46 may connect a vacuum splint dressing applied around the upper arm with a vacuum splint dressing applied around the forearm for fixation of the elbow joint at the desired angle during healing or rehabilitation.

It may be mentioned that a cover may be provided for sealing the accessory mount bracket 42 when accessories are not in use. Furthermore, it should be mentioned that the accessory bracket 42 is designed as a lead-through, provided with a screw thread, through one of the surface layers of the multilayer laminate, wherein an airtight laminate layer may be welded locally to the inside of said surface layer to isolate the passage through the accessory bracket from the granule-filled volume of the vacuum splint dressing.

The vacuum splint dressing according to the present invention may, in addition to the above-described embodiments, also be applied to e.g. an upper leg and/or a lower leg, shoulder dislocation patients and for spinal and/or pelvic fixation.

1. Vacuum splint dressing (1) comprising a multilayer laminate (2) forming an airtight volume partially filled with a filler of granules, wherein an evacuation and check valve (12) forms a sealable passage for air through one of the laminate layers, characterized in that the granules consist of bodies (20; 21) of expanded polypropylene (EPP).

2. Vacuum splint dressing according to claim 1, wherein the granule bodies (20; 21) are made of polypropylene with an original density in the order of 900 g/L, which is expanded in the ratio about 1/10 to about 1/20, preferably about 1/15.

3. Vacuum splint dressing according to claim 2, wherein the granule bodies (20; 21) are made of expanded polypropylene (EPP) with a density in the order of about 45 g/L to about 55 g/L, with an average density of about 50 g/L.

4. Vacuum splint dressing according to claim 1, wherein the granule bodies (20; 21) are non-spherical in the order of approx. 1 mm to approx. 5 mm, preferably approx. 1.5 mm to approx. 3 mm.

5. Vacuum splint dressing according to claim 1, wherein the multilayer laminate (2) comprises a first airtight surface layer (14), preferably made of polyurethane, a second airtight surface layer, preferably made of polyurethane (15), and an intermediate air-permeable layer (16), preferably in the form of a fabric made of nylon/polyamide or polyester, which is joined to the respective surface layer by means of a welded connection (18, 19).

6. Vacuum splint dressing according to claim 5, wherein the multilayer laminate (2) is joined by a doubled welded connection consisting of two welded joints (18, 19) which extend in parallel along the periphery of the multilayer laminate (17).

7. Vacuum splint dressing according to claim 5 comprising at least one tightening strap (5, 6) extending from either of the surface layers (14, 15), on which is arranged a piece of Velcro tape (7) which is attached to the tightening strap through a welded connection that runs around the Velcro tape and seals the surface behind the Velcro tape.

8. Vacuum splint dressing according to claim 1, wherein the check valve (12) comprises a valve holder part (37) which is permanently and airtightly anchored in an opening through one of the surface layers (14), a valve seat part (22) which is removably mounted in the valve holder part (37) through a screw connection (29, 38), a pump adapter part (30) which is removably mountable in the valve seat part (22) through a screw connection (35, 36), wherein the valve seat part (22) has a circular seat (23) for a circular valve membrane (24), wherein the valve membrane (24) is made of thermoplastic elastomer (TPE), preferably polyurethane plastic (TPU).

9. Vacuum splint dressing according to claim 8, comprising at least one accessory bracket (42) in the form of a coupling (38) accessible on the outside of the multilayer laminate (2) adapted for mounting accessories (43; 46) in addition to the check valve.

10. Vacuum splint dressing according to claim 9, wherein the accessory bracket (42) is designed as a lead-through provided with a screw thread through one of the surface layers of the multilayer laminate (2), and is provided with a cover with a seal for airtight closure of the lead-through when accessories are not used.

11. Vacuum splint dressing according to claim 9, wherein the accessory bracket (42) is designed as a lead-through provided with a screw thread through one of the surface layers of the multilayer laminate (2), wherein an airtight laminate layer that is welded to the inside of said surface layer isolates the lead-through from the granule-filled volume of the vacuum splint dressing.

12. Vacuum splint dressings according to claim 9, including accessories in the form of:
a height adjustable heel (43)
a shock-absorbing heel/sole (43),
a friction-increasing heel/sole (43),
a fixture brace (46) for a knee or an elbow joint,
a link (46) for connection between adjacent vacuum splint dressings.
